(19) United States
(12) Patent Application Publication
(10) Pub. No.: US 2020/0190562 A1
(43) Pub. Date: Jun. 18, 2020

(54) METHOD FOR DETECTING COPY NUMBER VARIATION

(71) Applicant: PHALANX BIOTECH GROUP, INC., Hsinchu (TW)

(72) Inventor: SHANG-CHI LIN, HSINCHU (TW)

(21) Appl. No.: 16/275,954

(22) Filed: Feb. 14, 2019

(30) Foreign Application Priority Data
Dec. 18, 2018 (TW) 107145537

(51) Int. Cl. C12Q 1/6816; G16B 20/10; G16B 20/20

(52) U.S. Cl. CPC C12Q 1/6816; C12Q 2600/156; G16B 20/20; G16B 20/10

(57) ABSTRACT

Provided is a method for detecting CNV including: (A) providing at least three test samples; (B) purifying nucleic acid from each test sample; (C) dividing all the nucleic acid samples into groups; (D) conducting whole genome amplification for each nucleic acid sample in the nucleic acid sample groups; (E) labelling the amplified nucleic acid samples with two fluorescent dyes; (F) performing hybridization on a chip that contains a set of specific human genome probes; (G) analyzing the signal data sets via locally weighted scatterplot smoothing (Lowess); (H) calibrating the signal data in view of corresponding probe values in a probe values set for calibration; (I) analyzing the calibrated results to obtain the CNV result of the test sample of interest. The detection method saves the reference sample and is beneficial for high-throughput detection.

### CROSS REFERENCE

This application claims priority under 35 U.S.C. § 119(a) to Taiwan Patent Application No. 107145537, filed on Dec. 18, 2018, the content of which is hereby incorporated by reference in its entirety.

### BACKGROUND OF THE INVENTION

### 1. Field of the Invention

The present invention relates to a method of genome detection, especially a method of Copy Number Variation (CNV) detection of cell chromosome.

### 2. Description of the Prior Arts

Traditionally, while using chromosome microarray chip to detect copy number variation, it is necessary to add both the test sample DNA and the reference sample DNA, which are labelled by different fluorescent dyes, to the same chip. Conventional fluorescent dyes are Cy3 (Cyanine Dye 3) and Cy5 (Cyanine Dye 5), which, after excitation, emit green light and red light, respectively. Then, the sample DNA is denatured into single strand DNA, followed by hybridization with the probes on a chromosome microarray chip and the comparison between fluorescent signals to identify whether the chromosomes of the test sample contain a gain or loss in a specific chromosomal region or not. Because a reference sample is required when detecting CNV in a traditional manner, it takes doubled reagents and human resources for every copy number variation detection, which results in a burden of detection.

Due to the high cost of traditional CNV detection methods in view of related reagents and human resources, a novel method with reduced cost for CNV detection is required.

### SUMMARY OF THE INVENTION

In order to overcome the shortcomings of the existing technology, the objective of the present invention is to spare the reference sample DNA and the related reagents. Besides, due to the lower standard deviation and the more convergent data, the present invention detects the CNV in a clearer manner.

To achieve the above objective, the present invention provides a method for detecting copy number variation, comprising (A) providing at least three test samples; (B) purifying nucleic acid from each test sample to obtain a respective nucleic acid sample for each test sample; (C) dividing all the nucleic acid samples into groups, wherein each group consists of two said nucleic acid samples, to obtain nucleic acid sample groups; (D) conducting whole genome amplification for each nucleic acid sample in the nucleic acid sample groups to obtain amplified nucleic acid samples in groups; (E) labelling one amplified nucleic acid sample in each group with a first fluorescent dye to obtain a first-fluorescent-dye-labelled amplified nucleic acid sample for each group, and labelling the other amplified nucleic acid sample in each group with a second fluorescent dye to obtain a second-fluorescent-dye-labelled amplified nucleic acid sample for each group; (F) mixing the first-fluorescent-dye-labelled amplified nucleic acid sample in each group with the second-fluorescent-dye-labelled amplified nucleic acid sample in said group to obtain a mixture, and conducting hybridization with the mixture on a chip that contains a set of human genome probes to obtain two signal data sets in one group for each chip, wherein each group of signal data sets consists of two signal data sets, and each of the signal data sets consists of signal data of all the probes against a labelled and amplified nucleic acid sample from each test sample; (G) analyzing the signal data sets in one group for each chip via locally weighted scatterplot smoothing (Lowess) to obtain two Lowess-analyzed signal data sets; (H) calibrating the signal data in the Lowess-analyzed signal data sets for the probes arranged in the order of genomic coordinates in view of corresponding probe values in a probe values set for calibration to obtain calibrated results, said probe values set for calibration is generated by: (i) using the signal data sets in groups derived from test samples in the same sample batch as the test sample of interest or in a different sample batch to (ii) obtain a probe values set for calibration via calculation in view of at least three Lowess-analyzed signal data sets, wherein said probe values set for calibration is a collection of probe values for calibration for all the probes; (I) analyzing the calibrated results to obtain a CNV result of the test sample of interest.

The present invention generates a probe value for calibration for each probe in view of at least three Lowess-analyzed signal data sets as a reference comparison value, and completes the CNV detection after calibrating the signal data of the test sample of interest. As compared to the traditional assays for detecting CNV, the present invention does not require a reference sample for each test sample of interest and thus reduces related reagents and human resources required. The present invention is advantageous to high-throughput CNV detection, because of the lower cost and economic benefit.

Preferably, the calculation in step (H)(ii) comprises: adjusting all of the at least three Lowess-analyzed signal data sets by mean centering based on the mean value of all the signal data of all the probes for Chromosome 1 to Chromosome 22, and calculating a median signal value for each probe on the chip based on the at least three Lowess-analyzed and mean center-adjusted signal data sets, wherein the median signal value for each probe on the chip is the probe value for calibration for each probe. The Lowess analysis reduces the cross interference between the two fluorescent dyes. The adjustment of mean centering reduces the signal intensity difference due to the difference in the amounts of nucleic acid in the hybridization as well as the difference in labelling efficiencies of the fluorescent dyes. Adopting the median of the signal data sets of the test samples for each probe excludes the outlier of signal data sets among the test samples resulting from experimental errors or deterioration of certain test samples. Such procedure generates the probe values set for calibration equivalent to the reference sample in the traditional CNV detection method.

Preferably, the calibration in step (H) comprises using the probe values set for calibration generated from the steps (i) and (ii) to conduct the calculation as follows:

log₂ (each probe signal data in the Lowess-analyzed signal data set of the test sample of interest/the corresponding probe value in the probe values set for calibration)

to obtain the log₂ ratio for each probe for the test sample of interest.

Preferably, after obtaining the log₂ ratio for each probe of the test sample of interest, the calibration in step (H) further comprises the following steps to obtain the calibrated results: adjusting the log₂ ratios for all the probes by zeroing the median of the ratios (that is to say, calculating the log₂ ratios for all the probes on the chip, and calculating the median based on all the log₂ ratios obtained, for example, if the median is −0.1, subtract −0.1 from all log₂ ratios such that the median of all obtained log₂ ratios becomes 0), calculating the median and standard deviation for each probe based on the median-zeroing-adjusted log₂ ratios of at least three consecutive probes arranged in the order of genomic coordinates. Calculating the medians based on the data sets of consecutive probes excludes the signal outlier within a sample resulting from experimental errors or a single invalid probe. Then, calculate the calibrated result as follows:

the median±the standard deviation for the corresponding probe of the test sample of interest×a coefficient.

If the median for a probe based on the median-zeroing-adjusted log₂ ratios is a positive value, calculate the result as:

the median−the standard deviation for the corresponding probe of the test sample of interest×a coefficient.

On the other hand, if the median for a probe based on the median-zeroing-adjusted log₂ ratios is a negative value, calculate the result as:

the median+the standard deviation for the corresponding probe of the test sample of interest×a coefficient.

In such manner, the deviated signals are converged in view of the standard deviation.

More preferably, the coefficient ranges from 0 to 1. Said coefficient adjusts the convergence level of the calibrated results as well as the background signal based on the standard deviation of the whole signal data sets and/or a standard sample of chromosome abnormality (for example, using the sample from Coriell Institute or the abnormality sample detected by a traditional CNV detection method) to highlight the gains or losses of fragments. More preferably, the coefficient ranges from 0.1 to 0.3. When the coefficient ranges from 0.3 to 0.5, the convergence level of the signal data of majority probes would be the highest (the standard deviation of the signal data sets is the lowest). When the coefficient ranges from 0 to 0.2, the background signal (not the signal of standard sample of chromosome abnormality supposed to be) would be the lowest.

Preferably, the analysis in step (I) is conducted by means including Circular binary segmentation (CBS), BioHMM, Forward-Backward Fragment-Annealing Segmentation or Wavelet smoothing.

Preferably, the first fluorescent dye in step (E) is Cy3 (Cyanine Dye 3) and the second fluorescent dye in step (E) is Cy5 (Cyanine Dye 5).

The term “zeroing the median” or “median-zeroing” used herein means calculating the median of all statistics samples and subtracting said median from each statistics sample value, so that the median value of all statistics samples becomes 0.

### BRIEF DESCRIPTION OF THE DRAWINGS

FIG. 1A is a flow chart of the CNV detection method of the present invention.

FIG. 1B is the continued flow chart of 1A.

FIG. 2A shows the result of the CNV detection of the present invention. Each spot in the figure represents a probe. The left end of the X-axis is chromosome 1, which is followed by chromosome 2 on the right side and so on. Chromosome 22 is followed by chromosome X on the right side and the chromosome X is followed by chromosome Y, which is at the right end of the X-axis.

FIG. 2B shows the result of the traditional CNV detection method. The test sample and reference sample are labelled with Cy5 fluorescent dye and Cy3 fluorescent dye, respectively, to conduct the assay on the same chromosome microarray chip. After Lowess analysis, plotting is based on the log₂ (red fluorescent signal/green fluorescent signal) of every probe.

FIG. 2C shows the result of the traditional CNV detection method. The test samples and reference sample are labelled with Cy5 fluorescent dye and Cy3 fluorescent dye, respectively, to conduct the assay on two different chromosome microarray chips. After Lowess analysis, plotting is based on the log₂ (red fluorescent signal/green fluorescent signal) of every probe.

### DETAILED DESCRIPTION OF THE PREFERRED EMBODIMENTS

In the following, the specific implantation of the CNV detection method of the present invention is explained through an embodiment. A person skilled in the art can easily understand the benefit and the effect of the present invention via the present specification and make various modifications and variations without departing from the scope and spirit of the present invention in order to implement and exercise the content of the present invention.

### Embodiment 1

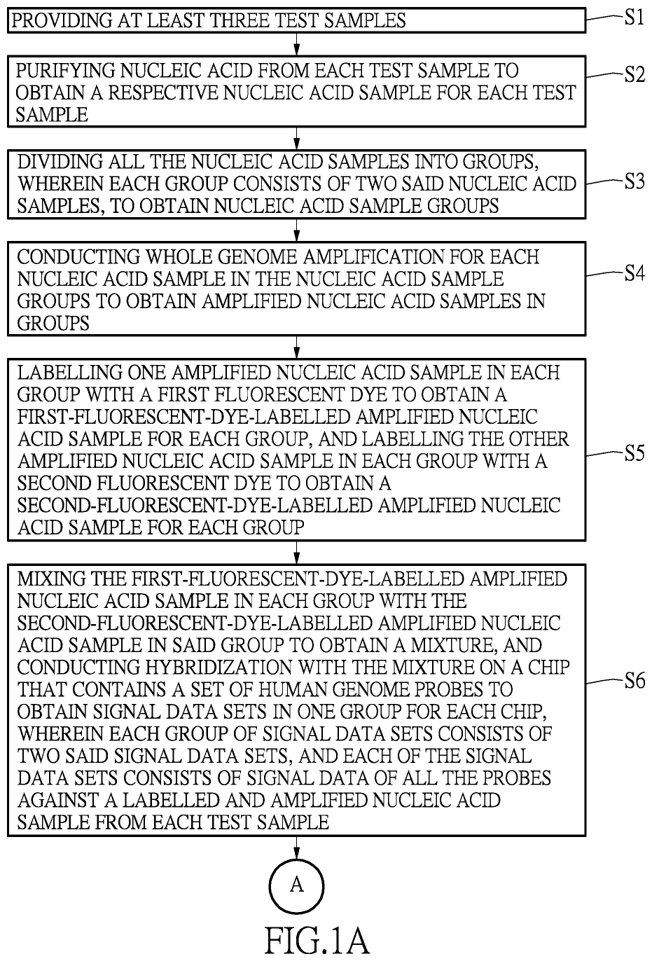

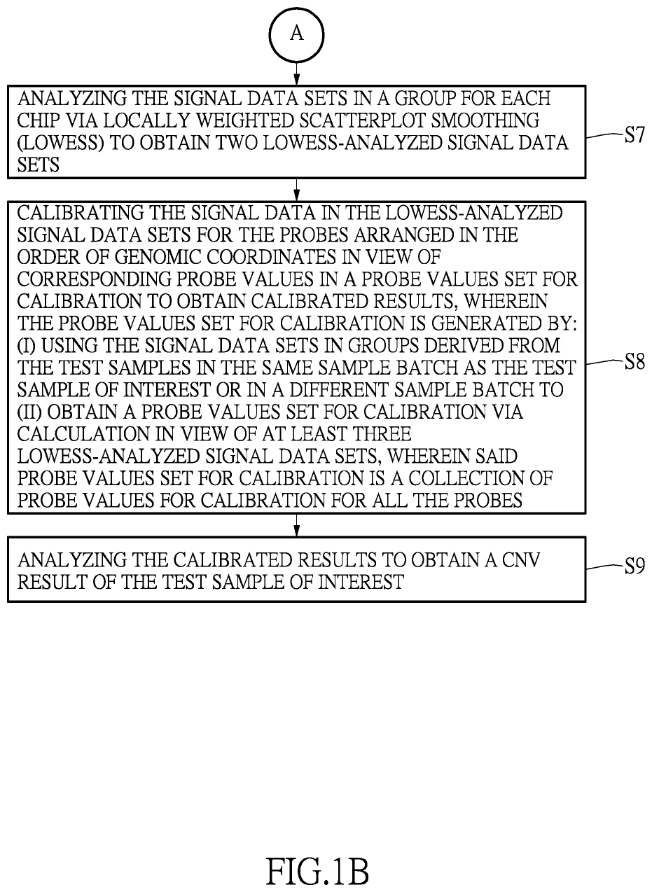

First, as shown in step (S1) of FIG. 1A, blood test samples from 20 patients with developmental delay are provided.

Then, as shown in step (S2) of FIG. 1A, nucleic acid is purified from each blood test sample to obtain a respective nucleic acid sample for each test sample. Particularly, MagPurix 12S System Automated Nucleic Acid Extraction System (Zinexts) and MagPurix Blood DNA extraction kit (Zinexts, cat # ZP02001-48) are used to extract DNA from each blood test sample. The purity of extracted DNA is assessed by the absorbance (O.D. value). The extracted DNA should meet the criteria O.D. 260/230>1.0 and O.D. 260/280>1.7.

As shown in step (S3) of FIG. 1A, all the nucleic acid samples are divided into groups, wherein each group consists of two said nucleic acid samples, to obtain nucleic acid groups. That is to say, the 20 DNA samples are randomly divided into groups of two, to obtain DNA sample groups. Every DNA sample group consists of two DNA samples. In other implementations, if the number of test samples is odd, the remaining ungrouped sample may be paired with the DNA sample from a traditional reference sample serving as another test sample or with any one of the grouped samples.

Next, as shown in step (S4) of FIG. 1A, whole genome amplification is conducted for each nucleic acid sample in the nucleic acid sample groups to obtain amplified nucleic acid samples in groups. Specifically, the whole genome amplification for each DNA sample in the DNA sample groups is conducted by using CytoOneArray Quick WGA labeling kit2.0 (Phalanx Biotech Group) and following the instructions in the user manual to obtain the amplified DNA samples in groups.

After that, as shown in step (S5) of FIG. 1A, one amplified nucleic acid sample in each group is labelled with a first fluorescent dye to obtain a first-fluorescent-dye-labelled amplified nucleic acid sample for each group, and the other amplified nucleic acid sample in each group is labelled with a second fluorescent dye to obtain a second-fluorescent-dye-labelled amplified nucleic acid sample for each group. Specifically, said labelling is carried out following the instructions in the user manual of the CytoOneArray Quick WGA labeling kit2.0 (Phalanx Biotech Group). One amplified DNA sample in each group is labelled with green fluorescent Cy3 (Cyanine Dye 3) followed by assessing the yield of the amplification and the labelling efficiency in view of the QC requirements recommended by the manufacturer to obtain a green-fluorescent-labeled amplified DNA sample for each group. Similarly, the other amplified DNA sample in each group is labelled with red fluorescent Cy5 (Cyanine Dye 5) followed by assessing the yield of the amplification and the labelling efficiency in view of the QC requirements recommended by the manufacturer to obtain a red-fluorescent-labelled amplified DNA sample for each group.

Then, as shown in step (S6) of FIG. 1A, the first-fluorescent-dye-labelled amplified nucleic acid sample in a group is mixed with the second-fluorescent-dye-labelled amplified nucleic acid sample in said group, followed by hybridization with the mixture on a chip that contains a set of human genome probes to obtain signal data sets in one group for each chip, wherein each group of signal data sets consists of two said signal data sets, and each of the signal data sets consists of signal data of all the probes against a labelled and amplified nucleic acid sample from a test sample. Specifically, the hybridization and washing are conducted as instructed by the manufacturer with the mixture of the green-fluorescent-labelled amplified nucleic acid sample in a group and the red-fluorescent-labelled amplified nucleic acid sample in said group, on a CytoOne Array v2.23 chip (Phalanx Biotech Group) with 32,816 probes (designed according to Hg19 database). After scanning by the Agilent scanner (G2565CA) with the scanning parameters recommended by the manufacturer, the chip image is analyzed by Genepix 6.0 software to obtain the raw data in gpr file format. Thus, for each chip, there are two signal data sets in one group, wherein each group of signal data sets consists of the signal data set of the first test sample and the signal data set of the second test sample (two signal data sets in total). Each of the signal data sets consists of signal data of all the probes against a labelled and amplified nucleic acid sample from a test sample.

Next, as shown in step (S7) of FIG. 1B, the signal data sets in one group for each chip are analyzed via locally weighted scatterplot smoothing (Lowess) to obtain two Lowess-analyzed signal data sets. Specifically, the signal data sets in one group for each chip are analyzed via Lowess, for the purpose of reducing the cross interference of the two fluorescences on each chip, to obtain a first Lowess-analyzed signal data set and a second Lowess-analyzed signal data set for each chip.

After that, as shown in step (S8) of FIG. 1B, the signal data in the Lowess-analyzed signal data sets for the probes arranged in the order of genomic coordinates is calibrated in view of the corresponding probe values in a probe values set for calibration to obtain calibrated results. The probe values set for calibration is generated by:

(i) using the signal data sets in groups derived from test samples in the same sample batch as the test sample of interest or in a different sample batch to

(ii) obtain a probe values set for calibration via calculation in view of at least three Lowess-analyzed signal data sets, wherein said probe values set for calibration is the collection of probe values for calibration for all the probes. Specifically, all of the 20 Lowess-analyzed signal data sets for all the probes (including those against Chromosome X and Chromosome Y) are adjusted by mean centering based on the mean value of all the signal data of all the probes against Chromosome 1 to Chromosome 22 in order to reduce the data reading error among the chips. In such manner, 20 Lowess-analyzed and mean centered signal data for each probe are obtained in accordance with the 20 Lowess-analyzed and mean center-adjusted signal data sets. Then, calculate the median signal value for each probe on the CytoOneArray v2.23 chip based on the 20 Lowess-analyzed and mean center-adjusted signal data sets, wherein the median signal value for each probe on said chip is the probe value for calibration for each probe. The collection of probe values for calibration for all the probes is the probe values set for calibration. Next, calibrate the signal data in the Lowess-analyzed signal data sets for the probes in view of corresponding probe values in the probe values set for calibration. In the present embodiment, the test sample of interest is one of the 20 test samples and the Lowess-analyzed signal data sets for the test sample of interest used to be calibrated in view of the probe values set for calibration is one of the 20 Lowess-analyzed signal data sets derived from the 20 test samples. However, in other embodiments, if there are new samples other than the 20 Lowess-analyzed samples, the new samples can be calibrated in view of the probe values set for calibration generated from the 20 Lowess-analyzed signal data sets derived from the 20 test samples of the present embodiment after finishing step (S1) to step (S7). Specifically, the calibration in view of the probe values set for calibration is calculated as follows:

log₂ (each probe signal data in the Lowess-analyzed signal data set of the test sample of interest/the corresponding probe value in the probe values set for calibration)

to obtain the log₂ ratio for each probe for the test sample of interest. Then, the log₂ ratios for all the probes for the test sample of interest are further adjusted by zeroing the median of the ratios. That is, after calculating the log₂ ratio for each probe for the test sample, subtract the median, calculated based on the log₂ ratios for the 32,816 probes, from the log₂ ratio for each probe. In this manner, the median of said 32,816 probes becomes zero. Next, arrange all the probes in the order of genomic coordinates and calculate the median and standard deviation for each probe based on the median-zeroing-adjusted log₂ ratios of 5 consecutive probes. In other words, calculate the median and standard deviation for probe 3 based on the ratios of probe 1 to probe 5; calculate the median and standard deviation for probe 4 based on the ratios of probe 2 to probe 6, and so on. After that, calculate as follows:

the median±the standard deviation for the corresponding probe of the test sample of interest×a coefficient

to obtain the calibrated result. If the median for a probe based on the median-zeroing-adjusted log₂ ratios is a positive value, calculate the result as:

the median−the standard deviation for the corresponding probe of the test sample of interest×a coefficient.

On the other hand, if the median for a probe based on the median-zeroing-adjusted log₂ ratios is a negative value, calculate the result as:

the median+the standard deviation for the corresponding probe of the test sample of interest×a coefficient.

In such manner, the deviated signals are converged in view of the standard deviation. Besides, the coefficient, which ranges from 0 to 1, adjusts the convergence level of the calibrated results based on the standard deviation of the whole signal data sets and a standard sample of chromosome abnormality to highlight the gains or losses of the fragment. In the present embodiment, the calibrated result for each probe is calculated with the coefficient being 0.2.

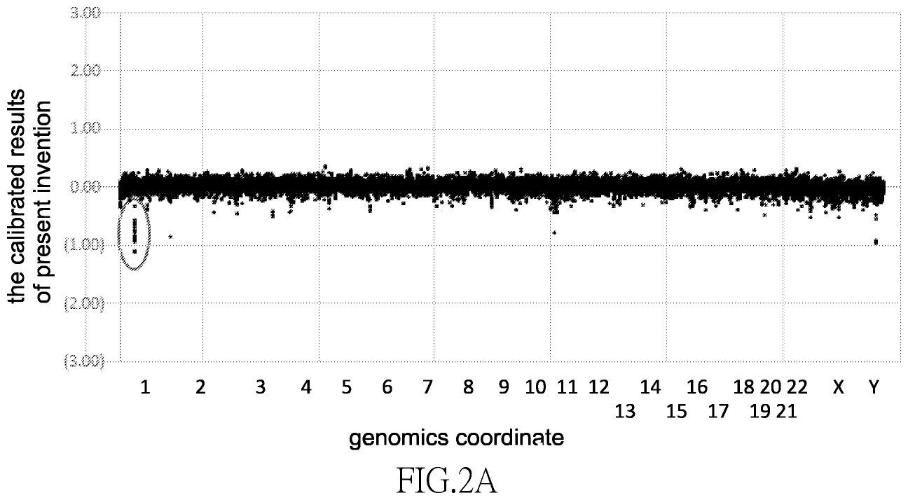

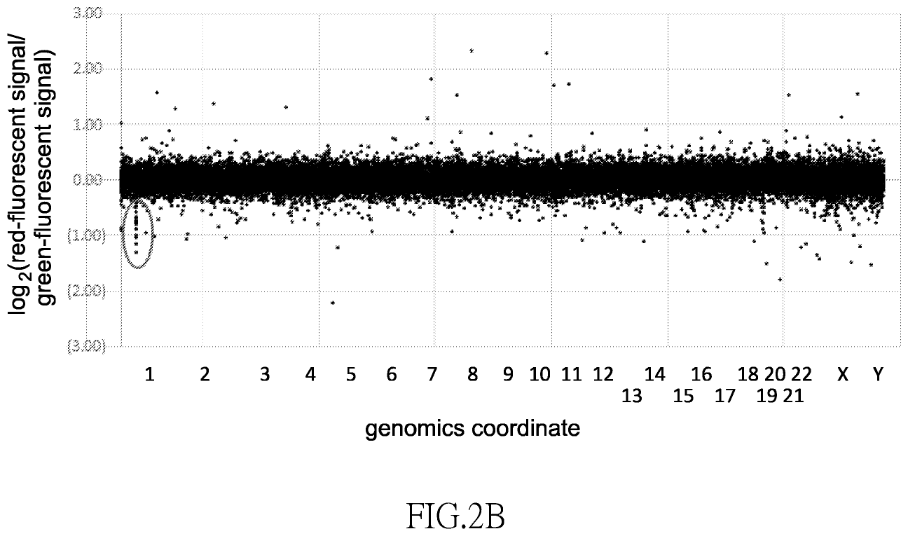

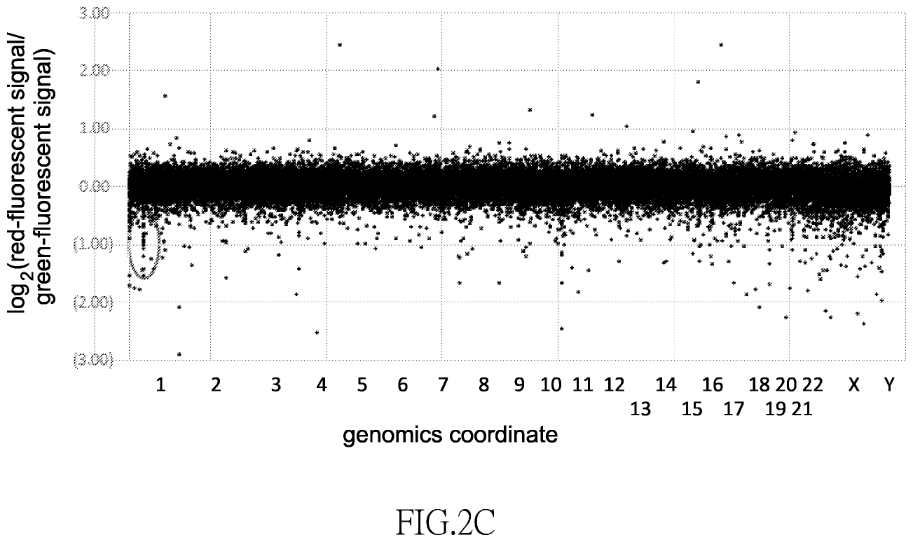

Next, plot the calibrated results on the Y-axis and the genomic coordinates on the X-axis for analysis (FIG. 2A). Finally, as shown in step (S9) of FIG. 1B, the CNV result of the test sample of interest is obtained via the analysis of the calibrated results: there is a loss of 2.23 Mb on Chromosome 1. In other embodiments, the Circular binary segmentation (CBS) can be used to automatically obtain the coordinate of CNV.

### Comparative Example 1

The experimental procedure in Comparative Example 1 is similar to Embodiment 1. However, in step (S1), only a DNA sample purified from a test sample is used in combination with a reference DNA sample (human genomic DNA, human male, promega cat # G1521). Hybridization is conducted with the mixture of the Cy5-fluorescent-labelled amplified DNA sample from the test sample and the Cy3-fluorescent-labelled amplified reference DNA sample on the same chip (Phalanx Biotech Group) with 32,816 probes. After Lowess analysis of the signal data of the test sample and the signal data of the reference sample, calculate the log₂ ratios as:

log₂ (signal datum of probe 1 in the Lowess-analyzed signal data set of the test sample/signal datum of probe 1 in the Lowess-analyzed signal data set of the reference sample)

and so on. After obtaining the log₂ ratios of all the 32,816 probes, each log₂ ratio is adjusted by mean-zeroing. Next, plot the adjusted results on the Y-axis and the genomic coordinates on the X-axis (FIG. 2B) to analyze the CNV result. The result in FIG. 2B and the result in FIG. 2A are from the same test sample, but are analyzed with different detection methods.

### Comparative Example 2

The experimental procedure is similar to Embodiment 1. However, DNA samples purified from two test samples (test sample A and test sample B) are used in combination with a reference DNA sample (human genomic DNA, human male, promega cat # G1521) in step (S1). The mixture of Cy5-labelled amplified DNA sample A and Cy3-labelled amplified reference sample is used to carry out a hybrdization on one chip (Phalanx Biotech Group) with 32,816 probes; and the mixture of Cy5-labelled amplified DNA sample B and Cy3-labelled amplified reference sample is used to carry out another hybridization assay on another chip (Phalanx Biotech Group) with 32,816 probes. After said two chips (chip A and chip B) are separately analyzed via Lowess, calculate the log₂ ratios as:

log₂ (signal datum of probe 1 in the Lowess-analyzed signal data set of the test sample A/signal datum of probe 1 in the Lowess-analyzed signal data set of the reference sample on chip B)

and so on. After obtaining the log₂ ratios of all the 32,816 probes, each log₂ ratio is adjusted by mean-zeroing. Next, plot the adjusted results on the Y-axis and the genomic coordinates on the X-axis (FIG. 2C) to analyze the CNV result. The result in FIG. 2C and the result in FIG. 2A are from the same test sample but are analyzed with different detection methods.

Comparing the result of the CNV detection method of the present invention (FIG. 2A) with the results of the traditional CNV detection methods (FIG. 2B and FIG. 2C), it can be seen that the present invention not only detects the loss of 2.23 Mb on Chromosome 1 as the traditional CNV detection methods but also reveals the copy number variation in a more obvious manner. The standard deviation of all the adjusted probe values, namely, the calibrated results, generated from the detection method of the present invention is 0.08. Generally speaking, the threshold of standard deviation for a successful analysis is 0.3. If the standard deviation is higher than 0.3, which means that the discrimination degree of the CNV detection is low, another CNV experiment or image analysis of the chips has to be conducted. The CNV detection method of the present invention shows better data convergence and a smaller standard deviation as compared to the traditional CNV detection methods showing a standard deviation of 0.17 for FIG. 2B (the test sample and the reference sample labelled with different fluorescent dyes on the same chip) as well as a standard deviation of 0.21 for FIG. 2C (the test sample and the reference sample labelled with different fluorescent dyes on two different chips).

In summary, the CNV detection method of the present invention saves a reference sample and related reagents and human resources for every test sample detection. The cost reducing effect is particularly obvious when conducting the CNV detections for a large sample size.

What is claimed is:

1. A method for detecting copy number variation (CNV), comprising:
(A) providing at least three test samples;
(B) purifying nucleic acid from each test sample to obtain a respective nucleic acid sample for each test sample;
(C) dividing all the nucleic acid samples into groups, wherein each group consists of two said nucleic acid samples, to obtain nucleic acid sample groups;
(D) conducting whole genome amplification for each nucleic acid sample in the nucleic acid sample groups to obtain amplified nucleic acid samples in groups;
(E) labelling one amplified nucleic acid sample in each group with a first fluorescent dye to obtain a first-fluorescent-dye-labelled amplified nucleic acid sample for each group, and labelling the other amplified nucleic acid sample in each group with a second fluorescent dye to obtain a second-fluorescent-dye-labelled amplified nucleic acid sample for each group;
(F) mixing the first-fluorescent-dye-labelled amplified nucleic acid sample in each group with the second-fluorescent-dye-labelled amplified nucleic acid sample in said group to obtain a mixture, and conducting hybridization with the mixture on a chip that contains a set of human genome probes to obtain signal data sets in one group for each chip, wherein each group of signal data sets consists of two said signal data sets, and each of the signal data sets consists of signal data of all the probes against a labelled and amplified nucleic acid sample from each test sample;
(G) analyzing the signal data sets in a group for each chip via locally weighted scatterplot smoothing (Lowess) to obtain two Lowess-analyzed signal data sets;
(H) calibrating the signal data in the Lowess-analyzed signal data sets for the probes arranged in the order of genomic coordinates in view of corresponding probe values in a probe values set for calibration to obtain calibrated results, wherein the probe values set for calibration is generated by:
(i) using the signal data sets in groups derived from the test samples in the same sample batch as the test sample of interest or in a different sample batch to
(ii) obtain a probe values set for calibration via calculation in view of at least three Lowess-analyzed signal data sets, wherein said probe values set for calibration is a collection of probe values for calibration for all the probes;
(I) analyzing the calibrated results to obtain a CNV result of the test sample of interest.

2. The method according to claim 1, wherein the calculation in step (H)(ii) comprises: adjusting all of the at least three Lowess-analyzed signal data sets by mean centering based on the mean value of all the signal data of all the probes for Chromosome 1 to Chromosome 22, and calculating a median signal value for each probe on the chip based on the at least three Lowess-analyzed and mean center-adjusted signal data sets, wherein the median signal value for each probe on the chip is the probe value for calibration for each probe.

3. The method according to claim 2, wherein the calibration in step (H) comprises using the probe values set for calibration generated from the steps (i) and (ii) to conduct the calculation as follows:
log₂ (each probe signal data in the Lowess-analyzed signal data set of the test sample of interest/the corresponding probe value in the probe values set for calibration)
to obtain the log₂ ratio for each probe for the test sample of interest.

4. The method according to claim 3, wherein after obtaining the log₂ ratio for each probe of the test sample of interest, the calibration in step (H) further comprises the following steps to obtain the calibrated results:
adjusting the log₂ ratios for all the probes by zeroing the median of the ratios,
calculating the median and standard deviation for each probe based on the median-zeroing-adjusted log₂ ratios of at least three consecutive probes arranged in the order of genomic coordinates, and
calculating the calibrated result as follows:
the median±the standard deviation for the corresponding probe of the test sample of interest×a coefficient.

5. The method according to claim 4, wherein the coefficient ranges from 0 to 1.

6. The method according to claim 5, wherein the coefficient ranges from 0.1 to 0.3.

7. The method according to claim 1, wherein the analysis in step (I) is conducted by means including Circular binary segmentation (CBS), BioHMM, Forward-Backward Fragment-Annealing Segmentation or Wavelet smoothing.

8. The method according to claim 1, wherein the first fluorescent dye in step (E) is Cy3 (Cyanine Dye 3) and the second fluorescent dye in step (E) is Cy5 (Cyanine Dye 5).
